(19) United States
(12) Patent Application Publication
(10) Pub. No.: US 2024/0000402 A1
(43) Pub. Date: Jan. 4, 2024

(54) MR-PET Apparatus with an Adjusting Unit for an Adjustment of a Position of a Plug-In Connecting Element and a Method for an Adjustment of a Position of a Plug-In Connecting Element

(71) Applicant: Siemens Healthcare GmbH, Erlangen (DE)

(72) Inventors: Johann Sukkau, Herzogenaurach (DE); Matthias Fenchel, Erlangen (DE)

(73) Assignee: Siemens Healthcare GmbH, Erlangen (DE)

(21) Appl. No.: 18/216,869

(22) Filed: Jun. 30, 2023

(30) Foreign Application Priority Data
Jun. 30, 2022 (EP) 22182181.2

(51) Int. Cl. A61B 6/04; G16H 10/20; A61B 6/00; A61B 6/03

(52) U.S. Cl. CPC A61B 6/0487; A61B 6/0407; G16H 10/20; A61B 6/465; A61B 6/037

(57) ABSTRACT

The disclosure is directed to an MR-PET apparatus with a patient table and a plug-in connecting unit having at least one plug-in connecting element arranged on the patient table. The at least one plug-in connecting unit may be designed to enter into a releasable connection with a corresponding plug-in connecting element of an external apparatus. A position of the at least one plug-in connecting element may be adjustable and the plug-in connecting unit has an adjusting unit for an independent adjustment of a target position of the at least one plug-in connecting element on the patient table.

### CROSS REFERENCE TO RELATED APPLICATIONS

This patent application claims priority to European Patent Application No. 22182181.2, filed Jun. 30, 2022, which is incorporated herein by reference in its entirety.

### BACKGROUND

### Field

The present disclosure relates to an MR-PET apparatus with a patient table and a plug-in connecting unit having at least one plug-in connecting element arranged on the patient table, wherein the at least one plug-in connecting element is designed to enter into a releasable connection with a corresponding plug-in connecting element of an external apparatus. The present disclosure also proceeds from a method for automatic adjustment of a position of at least one plug-in connecting element on a patient table.

### Related Art

Magnetic resonance-positron emission tomography (MR-PET) apparatuses are described in, for example, US20060293580 A1, which enable a combined and simultaneous recording of magnetic resonance signals and PET signals. However, if hardware components are situated in the beam path of PET photons, this causes a signal attenuation. A variety of approaches exist for taking account of such a signal attenuation in the context of a so-called attenuation correction. However, this does not usually take place completely, so that image artifacts can occur in the reconstructed PET mappings.

### BRIEF DESCRIPTION OF THE DRAWINGS/FIGURES

The accompanying drawings, which are incorporated herein and form a part of the specification, illustrate the embodiments of the present disclosure and, together with the description, further serve to explain the principles of the embodiments and to enable a person skilled in the pertinent art to make and use the embodiments.

FIG. 1 shows a schematic representation of an MR-PET apparatus according to one or more exemplary embodiments of the disclosure.

FIG. 2 shows a patient table with the at least one plug-in connecting element and an adjusting unit according to one or more exemplary embodiments of the disclosure.

FIG. 3 shows a method for an automatic adjustment of a target position of at least one plug-in connecting element according to one or more exemplary embodiments of the disclosure.

FIG. 4 shows a method for an automatic adjustment of a target position of at least one plug-in connecting element according to one or more exemplary embodiments of the disclosure.

FIG. 5 shows a plug-in connecting unit according to one or more exemplary embodiments of the disclosure.

The exemplary embodiments of the present disclosure will be described with reference to the accompanying drawings. Elements, features and components that are identical, functionally identical and have the same effect are—insofar as is not stated otherwise—respectively provided with the same reference character.

### DETAILED DESCRIPTION

In the following description, numerous specific details are set forth in order to provide a thorough understanding of the embodiments of the present disclosure. However, it will be apparent to those skilled in the art that the embodiments, including structures, systems, and methods, may be practiced without these specific details. The description and representation herein are the common means used by those experienced or skilled in the art to most effectively convey the substance of their work to others skilled in the art. In other instances, well-known methods, procedures, components, and circuitry have not been described in detail to avoid unnecessarily obscuring embodiments of the disclosure. The connections shown in the figures between functional units or other elements can also be implemented as indirect connections, wherein a connection can be wireless or wired. Functional units can be implemented as hardware, software or a combination of hardware and software.

An object of the present disclosure is to reduce any image artifacts in the reconstructed PET mappings.

The disclosure proceeds from an MR-PET apparatus with a patient table and a plug-in connecting unit having at least one plug-in connecting element arranged on the patient table, wherein the at least one plug-in connecting unit is designed to enter into a releasable connection with a corresponding plug-in connecting element of an external apparatus. According to the disclosure, a position of the at least one plug-in connecting element is adjustable, wherein the plug-in connecting unit has an adjusting unit for an independent adjustment of a target position of the at least one plug-in connecting element on the patient table.

The MR-PET (magnetic resonance-positron emission tomography) apparatus comprises a magnetic resonance apparatus and a positron emission tomography (PET) apparatus. The magnetic resonance apparatus comprises a scanner unit with a main magnet, a gradient coil unit and a high frequency antenna unit and is designed for acquiring magnetic resonance signals from a region of the patient that is to be examined. The PET apparatus has positron emission tomography detector modules (PET detector modules) with scintillation detector elements and photodiodes. In particular, the PET apparatus is designed for acquiring PET data.

In one or more exemplary embodiments, the MR-PET apparatus may include a patient positioning apparatus with a movable patient table. A patient, in particular a region of the patient that is to be examined, may be moved into and/or positioned by means of the movable patient table in a patient receiving region, in particular in a field of view (FOV) of the MR-PET apparatus for an MR-PET examination. For this purpose, the patient table has a positioning region for positioning and/or orientating the patient.

For an MR-PET examination, in particular for an MR examination, external apparatuses, in particular accessory units such as, for example, local high frequency coils are often arranged on the patient and/or on the patient table. Such a high frequency coil can be, for example, a head high frequency coil or a back high frequency coil, etc. For a connection of the external apparatus, in particular the accessory unit, to the MR-PET apparatus, in particular an evaluating unit of the MR-PET apparatus, a plug-in connecting unit with at least one plug-in connecting element is arranged on the patient table. The plug-in connecting unit can also comprise two or more plug-in connecting elements. For a connection of the external apparatus, in particular the accessory unit, to the MR-PET apparatus, in particular an evaluating unit and/or a controller of the MR-PET apparatus, the external apparatus, in particular the accessory unit, also has a plug-in connecting element corresponding to the at least one plug-in connecting element of the plug-in connecting unit. Therein, the at least one plug-in connecting element of the plug-in connecting unit can comprise a plug-in socket and the plug connecting element corresponding thereto of the external apparatus, in particular the accessory unit, can comprise a plug matched thereto. In an exemplary embodiment, the two plug-in connecting elements are parts of a common plug-in connection.

The plug-in connecting elements can comprise, in particular, one or more electrical and/or optical contacts for transferring, in particular, electrical and/or optical signals and/or in particular electrical and/or optical power. By way of the plug-in connecting unit, in particular the at least one plug-in connecting element, and the plug-in connecting element corresponding thereto of the external apparatus, a data exchange takes place between the external apparatus, for example, the local high frequency coil and an evaluating unit and/or a controller of the MR-PET apparatus. This data exchange can comprise, for example, acquired magnetic resonance data and/or control data for driving the external apparatus etc.

A releasable connection between the at least one plug-in connecting element of the plug-in connecting unit and the plug-in connecting element corresponding thereto of the external apparatus should therein be understood as a connection which is broken and/or suspended by separating the two plug-in connecting elements. By way of a renewed connection of the two plug-in connecting elements, the releasable connection can be re-created.

By way of the arrangement of the external apparatus on the patient and/or on the patient table for an MR-PET examination, additional material and/or objects can be situated in the beam path of the PET photons, in particular subregions of the external apparatus, which can lead to a weakening of the PET signal. In order to arrange these objects, in particular subregions of the external apparatus, outside the beam path of the PET photons, the position of the at least one plug-in connecting element of the plug-in connecting unit is designed so that it is adjustable on the patient table. In an exemplary embodiment, herein the plug-in connecting unit is a guiding element, for example, a guiding rail and/or a guiding groove in which the at least one plug-in connecting element is movably arranged so that the at least one plug-in connecting element can be moved, dependent upon the examination type and/or the region of the patient to be examined, out of the FOV of the MR-PET apparatus, in particular out of the FOV of the PET apparatus, for an MR-PET scan. Herein, the at least one plug-in connecting element of the plug-in connecting unit may be moved into a target position, wherein the target position is arranged outside the FOV of the MR-PET apparatus, in particular, an FOV of the PET apparatus.

Furthermore, the plug-in connecting unit has an adjusting unit which is designed for an independent and/or automatic adjustment of the target position of the at least one plug-in connecting element on the patient table. For this purpose, the adjusting unit can have a motor unit for generating a drive moment for moving the at least one plug-in connecting element into the target position. In addition, the adjusting unit can also have a controller and/or a computation unit for controlling the independent and/or automatic adjustment of the target position of the at least one plug-in connecting element on the patient table. The adjusting unit can therein take account of a current position of the at least one plug-in connecting element and/or a position of the patient table and/or an FOV of the PET scan and/or further parameters for the adjustment of the target position of the at least one plug-in connecting element.

By way of the disclosure, the plug-in connecting element can advantageously be moved into its target position in a particularly time-saving manner and thereby can be arranged and/or positioned outside the FOV of the PET apparatus. As a consequence thereof, undesirable artifacts and/or interfering influences in the PET image data can be minimized and also an image quality can advantageously be enhanced.

As compared with a manual adjustment which is undertaken before moving the patient into the patient receiving region, in which the user can estimate the target position only roughly, herein the automatic adjustment of the target position of the at least one plug-in connecting element can also only take place when the region to be examined is already arranged in the FOV of the MR-PET apparatus. By contrast therewith, in the case of a manual adjustment of the target position of the at least one plug-in connecting element, it can occur that for this purpose, the patient is moved several times into and out of the patient receiving region of the MR-PET apparatus and each time the position of the at least one plug-in connecting element must be set anew until it is no longer situated in the FOV, in particular of the PET apparatus, which however is very time-consuming.

A further advantage is that due to the time-saving positioning of the at least one plug-in element, a decay of a tracer and/or a radiophamaceutical even before the start of the MR-PET examination is avoided and therefore an additional radiation exposure in the patient is prevented.

In an advantageous development of the MR-PET apparatus according to the disclosure, it can be provided that the adjusting unit has a motor unit which is connected to the at least one plug-in connecting element. The motor unit is herein designed for generating a drive moment for a movement of the at least one plug-in connecting element, in particular within a guiding element of the adjusting unit. In an exemplary embodiment, the motor unit is designed to be magnetic resonance-compatible so that during a generation of a drive moment, an undesirable interaction between the motor unit and the magnetic resonance apparatus can advantageously be prevented. For example, the motor unit can comprise a magnetic resonance-compatible stepper motor, as described in the applications DE 10 2020 211 326 A1 or DE 10 2020 211 327 A1. Alternatively, or additionally, the motor unit can also comprise a pneumatic stepper motor and/or a piezo stepper motor and/or an electric stepper motor. The adjusting unit, in particular the motor unit also has a transfer element which transfers the drive moment generated by the motor unit to the at least one plug-in connecting element and/or connects the motor unit to the at least one plug-in connecting element. The transfer element may include a cable control for a particularly exact adjustment of a position of the at least one plug-in connecting element. Alternatively, the transfer element can also comprise a belt and/or a chain and/or a shaft, etc. By this means, in a constructionally simple manner, a drive moment can be provided for a movement, in particular an automatic and/or independent positioning, of the at least one plug-in connecting element of the plug-in connecting unit.

In an advantageous development of the MR-PET apparatus according to the disclosure, it can be provided that the motor unit is arranged at a foot end of the patient table. In an exemplary embodiment, the motor unit comprises a pneumatic stepper motor and/or a piezo stepper motor and/or an electric stepper motor. The foot end of the patient table therein comprises the end of the patient table which is arranged in a rear region of the patient table in the inward movement direction and/or during a movement of the patient table into the patient receiving region is moved last into the patient receiving region. During an MR-PET examination, the foot end of the patient table mostly protrudes out of the patient receiving region. By contrast, a head end of the patient table therein comprises the end of the patient table which is situated in a front region of the patient table in the inward movement direction and/or which, during a movement of the patient table into the patient receiving region, is moved first into the patient receiving region. In this way, an advantageous arrangement of the motor unit can be achieved in which an undesirable interaction with the magnetic resonance apparatus can be prevented.

In an advantageous development of the MR-PET apparatus according to the disclosure, it can be provided that the adjusting unit has a guiding element in which the at least one plug-in connecting element of the plug-in connecting unit is movably mounted, wherein the motor unit has a transfer element which is designed for a displacement of the at least one plug-in connecting element within the guiding element by at least 1.5 mm and by not more than 40 cm. The guiding element may include a guiding rail and/or a guiding groove, in which the at least one plug-in connecting element is mounted able to be moved, in particular in one direction, preferably in the longitudinal direction of the patient table. In an exemplary embodiment, the guiding element is arranged in a lateral region of the patient table adjoining a positioning region for positioning the patient. The guiding element may have a signal transmitting element which, in any settable position of the at least one plug-in connecting element, enables a transmission and/or an exchange of signals and/or data with an evaluating unit and/or a controller of the MR-PET apparatus. Therein, the signal transmitting element can be, for example, a cable, etc.

The transfer element of the motor unit may include a cable control. Therein, the maximum displacement of the at least one plug-in connecting element within the guiding unit can be limited by, for example, knots in the cable control. Alternatively, the maximum displacement of the at least one plug-in connecting element within the guiding unit can also be limited by, for example, a stop within the guiding element. In an exemplary embodiment, the maximum displacement of the at least one plug-in connecting element comprises 40 cm. In an exemplary embodiment, the maximum displacement of the at least one plug-in connecting element comprises 35 cm. In an exemplary embodiment, the maximum displacement of the at least one plug-in connecting element comprises 30 cm. In an exemplary embodiment, the maximum displacement of the at least one plug-in connecting element comprises 25 cm. In an exemplary embodiment, the maximum displacement of the at least one plug-in connecting element comprises 20 cm. The minimum displacement of the at least one plug-in connecting element results from the resolution of the PET detector.

By this means, advantageously, a simple displacement and/or positioning of the at least one plug-in connecting element into its target position can be enabled and thus can be arranged outside the FOV and/or the beam path of the PET apparatus during an MR-PET examination.

In an advantageous development of the MR-PET apparatus according to the disclosure, it can be provided that the adjusting unit has a guiding element in which the at least one plug-in connecting element of the plug-in connecting unit is movably mounted, wherein the motor unit has a transfer element which is designed for an adjustment of at least two different target positions of the at least one plug-in connecting element within the guiding element. The at least two different target positions can therein comprise predetermined target positions which are defined by way of the guiding element and/or by way of the transfer element. For example, the transfer element can comprise a cable control, wherein the two target positions can be defined by knots in the cable control. In an exemplary embodiment, the two different target positions comprise two end positions for the at least one plug-in connecting element which may be spaced from one another by not more than 40 cm. In an exemplary embodiment, the two different target positions comprise two end positions for the at least one plug-in connecting element which may be spaced from one another by not more than 35 cm. In an exemplary embodiment, the two different target positions comprise two end positions for the at least one plug-in connecting element which may be spaced from one another by not more than 30 cm. In an exemplary embodiment, the two different target positions comprise two end positions for the at least one plug-in connecting element which may be spaced from one another by not more than 25 cm. Particularly In an exemplary embodiment, the two different target positions comprise two end positions for the at least one plug-in connecting element which may be spaced from one another by not more than 20 cm. In this way, the at least one plug-in connecting element can particularly simply and reliably be arranged outside the FOV and/or the beam path of the PET apparatus during an MR-PET examination. In addition, in this way, a particularly simple and economical adjusting unit for the adjustment of the target positions of the at least one plug-in connecting element can be made available.

In an advantageous development of the MR-PET apparatus according to the disclosure, it can be provided that the motor unit has a force sensor for acquiring a tensile force acting upon the transfer element. The force sensor can have, for example, a strain gauge and/or other force sensors that a person skilled in the art deems useful for acquiring the tensile force acting upon the transfer element. During a positioning of the at least one plug-in connecting element, a tensile force acting on the transfer element should be substantially constant. If, however, a hindrance is present which makes displacement of the at least one plug-in connecting element more difficult, a tensile force acting upon the transfer element is thereby increased. For example, with a local high frequency coil plugged in, the cable between the local high frequency coil and the plug-in connecting element can become wound round the patient, which can result in injury to the patient. In addition, it can also be that the position of the patient covers a portion of the guiding rail. By means of the force sensor, the presence of a hindrance of this type can be detected. This also enables stopping of the positioning procedure of the at least one plug-in connecting element into the target position so that an injury to the patient and/or damage to components can be prevented. By this means, a higher safety standard can be provided in an automatic adjustment of the target position of the at least one plug-in connecting element.

In an advantageous development of the MR-PET apparatus according to the disclosure, it can be provided that the adjusting unit has a position detector, which is designed for detecting a position of the at least one plug-in connecting element. In the simplest case in which, for example, only two positions are available for adjustment of the target position of the at least one plug-in connecting element, such a position detector can comprise two photoelectric sensors. In addition, the position detector can also comprise a magnetic resonance-compatible encoder which is designed for detecting an absolute position and/or velocity and/or direction of the movement of the at least one plug-in connecting element. In an exemplary embodiment, the encoder comprises an optical encoder. In addition, further position detectors deemed useful by a person skilled in the art are readily conceivable. By this means, a simple and automatic detection of the position, in particular a current position, of the at least one plug-in connecting element can be achieved. In particular, herein, the position can be detected even before the adjustment of the target position of the at least one plug-in connecting element, as well as the target position of the at least one plug-in connecting element.

In an advantageous development of the MR-PET apparatus according to the disclosure, it can be provided that the adjusting unit has a guiding element in which the at least one plug-in connecting element is movably mounted, wherein the position detector is arranged on the guiding element. By means of the detection of the current position of the at least one plug-in connecting element directly on the guiding element and thus on at least one plug-in connecting element, advantageously, inaccuracies in the transfer element of the motor unit in the detection of the position of the at least one plug-in connecting element can be prevented. If the transfer element comprises, for example, a cable control, on detection of the position close to the motor unit, a stretching of the cable control could lead to inaccuracies in the detected position, which is advantageously prevented by the arrangement of the position detector on the guiding element.

In an advantageous development of the MR-PET apparatus according to the disclosure, it can be provided that the adjusting unit has a controller which is designed, on the basis of current position data relating to the at least one plug-in connecting element and on the basis of at least one examination parameter, to establish a target position for the at least one plug-in connecting element. In an exemplary embodiment, the current position of the at least one plug-in connecting element is provided by the position detector. The at least one examination parameter can comprise, for example, a position of the patient table which it is to assume during the MR-PET examination. Alternatively, or additionally, the at least one examination parameter can also comprise a position and/or a type and/or a number of the external apparatuses connected to the at least one plug-in connecting element, in particular local high frequency coils. Alternatively, or additionally, the at least one examination parameter can also comprise an FOV and/or a position of the beam path of the PET apparatus in relation to an examination position of the patient table and/or further parameters deemed useful by a person skilled in the art.

The controller according to the disclosure comprises at least one computation module and/or a processor. Thus, the controller, in particular, is configured to carry out computer-readable instructions in order to establish the target position for the at least one plug-in connecting element. In particular, the controller comprises a storage unit, wherein computer-readable information is stored on the storage unit, wherein the controller is designed to load the computer-readable information from the storage unit and to execute the computer-readable information.

The components of the controller can be designed mainly in the form of software components. In principle, these components can also be realized partially, in particular, if particularly rapid calculations are involved, in the form of software-supported hardware components, for example, FPGAs or suchlike. Similarly, the required interfaces can be designed, for example, where only an acceptance of data from other software components is concerned, as software interfaces. However, they can also be designed as interfaces which are constructed as hardware and are controlled by suitable software. It is naturally also conceivable that a plurality of the aforementioned components is realized grouped together in the form of an individual software component and/or a software-supported hardware component.

This design of the disclosure has the advantage that an established target position of the at least one plug-in connecting element can be provided particularly rapidly and effectively for automatic adjustment of the target position.

In an advantageous development of the MR-PET apparatus according to the disclosure, it can be provided that the controller is designed to control an automatic adjustment of the target position of the at least one plug-in connecting element. In an exemplary embodiment, herein the controller is designed for controlling the position detector and the adjusting unit. In this way, a particularly rapid and effective adjustment of the target position of the at least one plug-in connecting element for the planned MR-PET examination can be achieved.

The disclosure also proceeds from a method for automatic adjustment of a target position of at least one plug-in connecting element on a patient table by means of an adjusting unit of an MR-PET apparatus, comprising the following method steps:
providing a current position information item of the at least one plug-in connecting element,
providing at least one examination parameter,
establishing the target position of the at least one plug-in connecting element on the basis of the current position information item and the at least one examination parameter, and
adjusting the target position of the at least one plug-in connecting element.

In an exemplary embodiment, the provision of the current position information item of the at least one plug-in connecting element takes place automatically and/or independently by way of the position detector of the adjusting unit. The current position information item is therein advantageously provided to the controller of the adjusting unit, wherein the controller controls a detection of the current position information.

In an exemplary embodiment, the provision of the at least one examination parameter takes place automatically and/or independently by way of the controller of the adjusting unit. Herein, the controller can access the examination parameters stored in the MR-PET apparatus. The at least one examination parameter can comprise, for example, a position of the patient table which it is to assume during the MR-PET examination. Alternatively, or additionally, the at least one examination parameter can also comprise a position and/or a type and/or a number of the external apparatuses connected to the at least one plug-in connecting element, in particular local high frequency coils. Alternatively, or additionally, the at least one examination parameter can also comprise an FOV and/or a position of the beam path of the PET apparatus in relation to an examination position of the patient table and/or further parameters deemed useful by a person skilled in the art.

In an exemplary embodiment, the establishment of the target position of the at least one plug-in connecting element takes place automatically and/or independently by way of the controller of the adjusting unit. In an exemplary embodiment, herein the controller comprises a corresponding software item and/or algorithm for determining the target position for at least one plug-in connecting element.

In an exemplary embodiment, the establishment of the target position of the at least one plug-in connecting element takes place automatically and/or independently by way of the adjusting unit, wherein for this purpose, the controller of the adjusting unit drives the motor unit of the adjusting unit accordingly.

By way of the disclosure, the plug-in connecting element can advantageously be moved into its target position in a particularly time-saving manner and thereby can be arranged and/or positioned outside the FOV of the PET apparatus. As a consequence thereof, undesirable artifacts and/or interfering influences in the PET image data can be minimized and also an image quality can advantageously be enhanced.

As compared with a manual adjustment which is undertaken before moving the patient into the patient receiving region, wherein the user can estimate the target position only roughly, herein the automatic adjustment of the target position of the at least one plug-in connecting element can also take place only when the region to be examined is already arranged in the FOV of the MR-PET apparatus. By contrast therewith, in the case of a manual adjustment of the target position of the at least one plug-in connecting element, it can occur that for this purpose, the patient is moved several times into and out of the patient receiving region of the MR-PET apparatus and each time the position of the at least one plug-in connecting element must be set anew until it is no longer situated in the FOV, in particular of the PET apparatus, which however is very time-consuming.

A further advantage is that, due to the time-saving positioning of the at least one plug-in element, a decay of a tracer and/or a radiophamaceutical even before the start of the MR-PET examination is avoided and therefore an additional radiation exposure in the patient is prevented.

The advantages of the method according to the disclosure substantially correspond to the advantages of the MR-PET apparatus according to the disclosure, as described in detail above. Features, advantages or alternative embodiments mentioned herein can also be transferred to the other embodiments and vice versa.

In an advantageous development of the method according to the disclosure, it can be provided that before the adjustment of the target position of the at least one plug-in connecting element, a safety questionnaire is generated and is output to a user by means of a user interface. The safety questionnaire can comprise, for example, an information item regarding the target position and can impart to the user that by means of a confirmation input, an adjustment and/or positioning of the at least one plug-in connecting element is started in this target position.

In an exemplary embodiment, the user interface comprises an output unit, in particular an optical output unit, for example, a monitor and/or a display. Furthermore, the user interface also comprises an input unit such as a keyboard and/or a computer mouse.

In this way, a high safety standard for the adjustment and/or positioning of the at least one plug-in connecting element in this target position can be provided. In particular, by this means, the user can check again whether the established target position of the at least one plug-in connecting element is arranged outside the FOV of the PET apparatus and/or matches the planned MR-PET examination.

In an advantageous development of the method according to the disclosure, it can be provided that the adjustment of the target position of the at least one plug-in connecting element is detected by a camera and is output to the user by means of a user interface. In an exemplary embodiment, the camera is arranged such that a field of view of the camera, the FOV of the MR-PET apparatus and, in particular the at least one plug-in connecting element and the guiding unit in which the at least one plug-in connecting element is movably mounted is acquired. For this purpose, the camera can be arranged within the patient receiving region or also outside the patient receiving region.

This design of the disclosure enables an advantageous monitoring of the adjustment of the target position by the user. In particular, in case of unexpected hindrances or occurrences during the adjustment of the target position, the user can intervene and thus also advantageously prevent injuries to the patient and/or damage to hardware components.

In an advantageous development of the method according to the disclosure, it can be provided that during the adjustment of the target position of the at least one plug-in connecting element, the adjustment process can be ended manually. In an exemplary embodiment, herein the MR-PET apparatus, in particular the user interface, has an emergency stop switch. The emergency stop switch can comprise, for example, a pop-up window which is displayed for the user by means of the user interface during the adjustment process. This pop-up window can therein comprise an emergency stop button. In an exemplary embodiment, a pop-up window of this type is generated and provided by the controller.

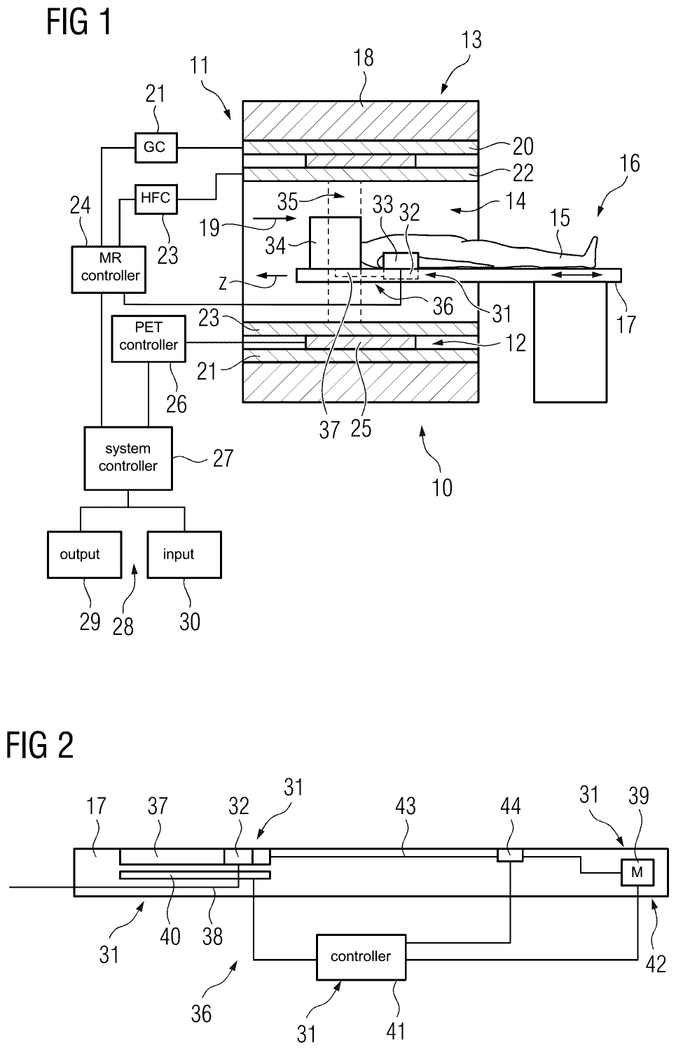

FIG. 1 shows a schematic representation of an embodiment of an MR-PET apparatus 10 according to the disclosure. The MR-PET apparatus 10 comprises a magnetic resonance apparatus 11 and a positron emission tomography apparatus (PET apparatus 12).

The magnetic resonance apparatus 11 comprises a scanner 13 formed by a magnet unit and a patient receiving region 14 surrounded by the scanner 13 for receiving a patient 15. In the present exemplary embodiment, the patient receiving region 14 is designed cylindrical and is surrounded cylindrically in a circumferential direction by the scanner 13, in particular the magnet unit. The patient 15 can be pushed and/or moved by means of a patient positioning apparatus 16 of the MR-PET apparatus 10 into the patient receiving region 14. For this purpose, the patient positioning apparatus 16 has a patient table 17 which is designed to be movable within the patient receiving region 14. In particular, the patient table 17 is mounted to be movable in the direction of a longitudinal extent of the patient receiving region 14 and/or in the z-direction.

The scanner 13, in particular the magnet unit, comprises a superconducting main field magnet 18 for generating a strong and, in particular, constant main magnetic field 19. Furthermore, the scanner 13, in particular the magnet unit, has a gradient coil unit 20 for generating magnetic field gradients that are used for position encoding during an imaging process. The gradient coil unit 20 is controlled by means of a gradient controller 21 of the magnetic resonance apparatus 11. The scanner 13, in particular, the magnet unit further comprises a high frequency antenna unit 22 for exciting a polarization which forms in the main magnetic field 19 generated by the main field magnet 18. The high frequency antenna unit 22 is controlled by a high frequency antenna controller 23 of the magnetic resonance apparatus 11 and radiates high frequency magnetic resonance sequences into the patient receiving region 14 of the magnetic resonance apparatus 11.

For controlling the main field magnet 18, the gradient controller 20 and, for controlling the high frequency antenna controller 22, the magnetic resonance apparatus 11 has a magnetic resonance (MR) controller 24. The magnetic resonance controller 24 controls the magnetic resonance apparatus 11, for example, the execution of a pre-determined imaging gradient echo sequence, centrally. Furthermore, the magnetic resonance controller 24 comprises an evaluating unit (not shown in detail) for evaluating medical magnetic resonance image data.

The magnetic resonance apparatus 11 described can naturally comprise further components that magnetic resonance devices 11 typically have. A general mode of operation of a magnetic resonance apparatus 11 is also known to a person skilled in the art, so that a detailed description of the general components is not included.

The PET apparatus comprises a plurality of positron emission-tomography detector modules (PET detector modules 25) which are arranged in a ring form and surround the patient receiving region 14 in the peripheral direction. The PET detector modules 25 each have a plurality of positron emission tomography detector elements (PET detector elements) (not shown in detail) which are arranged as a PET detector array which comprises a scintillation detector array with scintillator crystals, for example LSO crystals. Furthermore, the PET detector modules 25 each comprise a photodiode array, for example, an avalanche photodiode array or an APD photodiode array which are arranged downstream of the scintillation detector array within the PET detector modules 25.

By means of the PET detector modules 25, photon pairs which result from the annihilation of a positron with an electron are detected. Trajectories of the two photons enclose an angle of 180°. In addition, the two photons each have an energy of 511 keV. The positron is emitted by a radiopharmaceutical which is administered to the patient 15 by means of an injection. On passing through material in the beam path, PET photons arising from the annihilation can be attenuated, wherein the attenuation probability depends on the path length through the material and the corresponding attenuation coefficient of the material.

In addition, the PET detector modules 25 each have a detector electronics unit which comprises an electric amplifier circuit and further electronic components (not shown in detail).

For controlling the detector electronics unit and the PET detector modules 25, the MR-PET apparatus 10, in particular the PET apparatus 12, has a PET controller 26. The PET controller 26 controls the PET apparatus 12 centrally. Furthermore, the PET controller 26 comprises an evaluating unit for evaluating PET data. The controller 26 may include processing circuitry that is configured to perform one or more operations and/or functions of the controller 26. The PET apparatus 12 described can naturally comprise further components that PET apparatuses 12 typically have. A general mode of operation of a PET apparatus 12 is also known to a person skilled in the art, so that a detailed description of the further components is not included.

The MR-PET apparatus 10 also comprises a central computer (system controller) 27 which, for example, matches a detection and/or an evaluation of magnetic resonance signals and PET signals to one another. The computer 27 can be a central system controller. The controller 27 may include processing circuitry that is configured to perform one or more operations and/or functions of the controller 27.

Furthermore, the MR-PET apparatus 10 comprises a user interface 28 which is connected to the system controller 27. Control information such as, for example, image data can be displayed on an output unit 29, for example, on at least one monitor of the user interface 28 for medical operating personnel. In addition, the user interface 28 has an input unit 30 by means of which the information and/or parameters can be input by the medical operating personnel during a scanning procedure.

The patient table 17 also has a plug-in connecting unit 31, wherein the plug-in connecting unit 31 has at least one plug-in connecting element (plug-in connector) 32 arranged on the patient table 17. The plug-in connecting unit 31 can therein comprise two or more plug-in connecting elements 32. Herein, the disclosure is explained in more detail on the basis of a single plug-in connecting element 32. The plug-in connecting element 32 is designed to enter into a releasable connection with a plug-in connecting element (plug-in connector) 33 of an external apparatus 34 corresponding to the plug-in connecting element 32 of the plug-in connecting unit 31. The external apparatus 34 comprises, for example, an accessory unit such as, for example, a local high frequency coil.

The plug-in connecting element 32 of the plug-in connecting unit 31 may be designed as a plug-in socket. The plug-in connecting element 33 of the external apparatus 34 is designed as a plug corresponding thereto.

For an MR-PET examination, the patient table 17 is moved into the patient receiving region 14 together with the patient 15 and the further units arranged and/or positioned on the patient 15 and/or on the patient table 17, such as for example, a local high frequency coil in the patient receiving region 14 until the region of the patient 15 to be examined is situated in the FOV of the MR-PET apparatus. It can therein also be the case that the plug-in connecting element 32 of the plug-in connecting unit 31 is arranged within an FOV 35 of the PET apparatus 12. In order to prevent this, a position of the plug-in connecting element 32 of the plug-in connecting unit 31 is designed to be adjustable. For this purpose, the plug-in connecting unit 31 has an adjusting unit 36 which is designed for an independent and/or automatic adjustment of a target position of the plug-in connecting element 32 on the patient table 17.

FIG. 2 shows the patient table 17 with the plug-in connecting unit 31, in particular the plug-in connecting element 32 and the adjusting unit 36 in more detail.

For an adjustable target position of the plug-in connecting element 31 of the plug-in connecting unit 31, the adjusting unit 36 has a guiding element 37 in which the plug-in connecting element 32 is movably mounted. The guiding element 37 is designed as a guiding rail and/or as a guiding groove in which the plug-in connecting element 32 is mounted to be movable in one direction, preferably the longitudinal direction of the patient table 17. The guiding element 37 can therein have a T-shaped and/or dovetail-shaped cross-section in order to ensure a reliable guidance of the plug-in connecting element 32. In addition, the adjusting unit 36 has a contact element 38 and/or a signal transmitting element which transmits signals and/or data between the movably mounted plug-in connecting element 32 to an evaluating unit of the magnetic resonance controller 24. The contact element 38 can therein comprise a wound cable and/or a spiral cable which can compensate for a movement of the plug-in connecting element 32. In addition, the contact element 38 can comprise a contact plate which is arranged within the guiding element 37 and/or further contact elements 38 as deemed useful by the person skilled in the art. The controller 24 may include processing circuitry that is configured to perform one or more operations and/or functions of the controller 24.

For the independent and/or automatic adjustment of the target position of the plug-in connecting element 32, the adjusting unit (adjuster) 36 also has a motor unit (e.g., motor) 39, a position detector 40 and a controller 41. The motor unit 39 may be designed magnetic resonance-compatible. For example, the motor unit 39 can comprise a magnetic resonance-compatible stepper motor, as described in the applications DE 10 2020 211 326 A1 or DE 10 2020 211 327 A1. Alternatively, or additionally, the motor unit 39 can also comprise a pneumatic stepper motor and/or a piezo stepper motor and/or an electric stepper motor. In addition, the motor unit 39 is arranged on a foot end 42 of the patient table 17, wherein the motor unit 39 herein may include a pneumatic stepper motor and/or a piezo stepper motor and/or an electric stepper motor. The adjusting unit 36, in particular the motor unit 39 also has a transfer element 43 which transfers the drive moment generated by the motor unit 39 to the plug-in connecting element 32 and/or connects the motor unit 39 to the plug-in connecting element 32. The transfer element 43 may include a cable control for a particularly exact adjustment of a position of the at least one plug-in connecting element 32. The controller 41 may include processing circuitry that is configured to perform one or more operations and/or functions of the controller 41.

The transfer element 43 is designed for displacement of the plug-in connecting element 32 into the target position by at least 1.5 mm and not more than 40 cm. In an exemplary embodiment, the maximum displacement of the at least one plug-in connecting element 32 is 35 cm. In an exemplary embodiment, the maximum displacement of the at least one plug-in connecting element 32 is 30 cm. In an exemplary embodiment, the maximum displacement of the at least one plug-in connecting element 32 is 25 cm. In an exemplary embodiment, the maximum displacement of the at least one plug-in connecting element 32 is 20 cm. The minimum displacement of the at least one plug-in connecting element 32 results from the resolution of the PET detector module 25.

In addition, the transfer element 43 can also be designed for an adjustment of at least two different target positions of the plug-in connecting element 32 within the guiding element 37. If, for example, the transfer element 43 comprises a cable control, then the two target positions can be defined by knots in the cable control. Therein, the two different target positions can comprise two end positions which may be spaced from one another by not more than 40 cm. In an exemplary embodiment, the two target positions, in particular the end positions, are arranged spaced not more than 35 cm from one another. In an exemplary embodiment, the two target positions, in particular the end positions, are arranged spaced not more than 30 cm from one another. In an exemplary embodiment, the two target positions, in particular the end positions, are arranged spaced not more than 25 cm from one another. In an exemplary embodiment, the two target positions, in particular the end positions, are arranged spaced not more than 20 cm from one another.

The motor unit 39 additionally has a force sensor 44 for a detection of a tensile force acting upon the transfer element 43. The force sensor 44 comprises, for example, a strain gauge. By means of the force sensor 44, it can be detected if during a positioning of the plug-in connecting element 32 into a target position, a hindrance is present that would require an increased tensile force to move the plug-in connecting element 32.

The position detector 40 is designed for detecting a position of the plug-in connecting element 32. For this purpose, the position detector 40 is arranged on the guiding element 37. The position detector 40 can comprise, for example, photoelectric sensors, which is advantageous if the adjusting unit 36 is designed for adjustment of only defined, preferably two target positions. If, however, the adjusting unit 36 is designed for adjustment of any desired target position within the guiding unit 37, the position detector 40, by contrast, may include an encoder, in particular a magnetic resonance-compatible encoder which is designed for detecting an absolute position and/or velocity and/or direction of the movement of the at least one plug-in connecting element 32. In an exemplary embodiment, the magnetic resonance-compatible encoder comprises an optical encoder.

The controller 41 of the adjusting unit 36 is designed, on the basis of current position data from the plug-in connecting element 32 and on the basis of at least one examination parameter, to establish a target position for the plug-in connecting element 32. The controller 41 is also designed to control an automatic adjustment of the target position of the plug-in connecting element 32. For this purpose, the controller 41 has a corresponding control software and/or evaluating software. The corresponding control software and/or evaluating software can be stored in a storage unit of the controller 41 or in an external storage unit.

The current position data of the plug-in connecting element 32 is provided by the position detector 40 of the controller 41. The at least one examination parameter can comprise, for example, a position of the patient table 17 which it is to assume during the MR-PET examination. Alternatively, or additionally, the at least one examination parameter can also comprise a position and/or a type and/or a number of the external apparatuses 34 connected to the at least one plug-in connecting element 32, in particular, local high frequency coils. Alternatively, or additionally, the at least one examination parameter can also comprise an FOV 35 and/or a position of the beam path of the PET apparatus 12 in relation to an examination position of the patient table 17 and/or further parameters deemed useful by a person skilled in the art.

FIG. 5 shows an alternative exemplary embodiment of the plug-in connecting unit 300. In principle, components, features and functions that remain substantially the same are identified with the same reference signs. The following description is essentially restricted to the differences from the exemplary embodiment in FIGS. 1 and 2, wherein with regard to components, features and functions which remain the same, reference can be made to the description of the exemplary embodiment shown in FIGS. 1 and 2.

FIG. 5 shows the patient table 17 with a plug-in connecting unit 300, wherein the plug-in connecting unit 300 has two plug-in connecting elements 301 and an adjusting unit 302. Each of the plug-in connecting elements 301 is designed for a releasable connection to a corresponding plug-in connecting element of an external apparatus, for example, of a local high frequency coil. The adjusting unit 302 has a motor unit 303 and a transfer element 304. The motor unit 303 is arranged on the foot end 42 of the patient table 17 and, in the present exemplary embodiment, comprises a pneumatic cylinder. The pneumatic cylinder is formed, for example, from a plastics material. In addition, the motor unit 303 has an air pump and two air valves for a reversal of a direction of movement of the transfer element 304 moved and/or driven by means of the motor unit 303.

The transfer element 304 comprises a cable control which is connected to the two plug-in connecting elements 301 of the plug-in connecting unit 300. The adjusting unit 302 also has rollers 305, wherein the cable control is mounted and/or guided and/or deflected by means of the rollers 305, in the present exemplary embodiment, by means of six rollers 305. In the present exemplary embodiment, the cable control comprises a cable made of aramid fibers. In principle, other materials can be used for designing the cable of the cable control. The cable forms a closed loop. By way of a cable knot 306, the cable is connected to the cylinder of the pneumatic motor unit 303. In addition, the cable is connected via a cable knot 306 to the movable plug-in connecting element 301.

The plug-in connecting unit 300 also has two guiding elements 307, which are arranged at the head end 308, in particular at a lateral region of the head end 308 of the patient table 17. The guiding elements 307 also each have a position detector (not shown in detail in FIG. 5), for example, a position encoder. During a movement of the pneumatic cylinder, via the cable knots 306, the cable is conjointly moved and via the two further cable knots 306, the two plug-in connecting elements 301 are also conjointly moved. Thereby, a positional displacement of the two plug-in connecting elements 301 in the z-direction also takes place. A further embodiment of the plug-in connecting unit 300 shown in FIG. 5 corresponds to the description relating to FIG. 2.

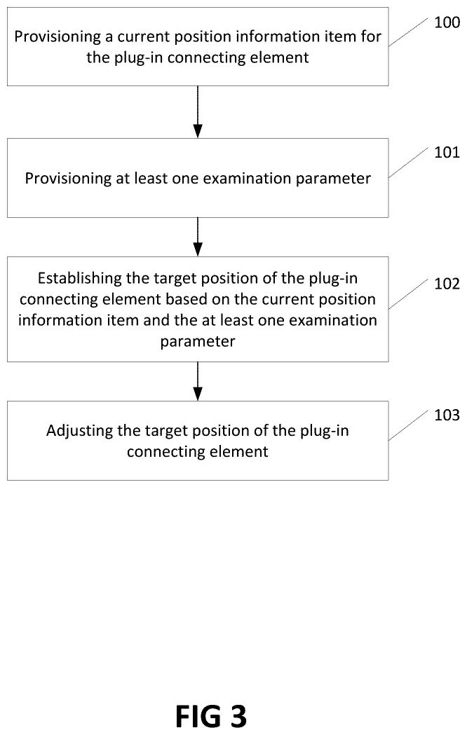

FIG. 3 shows a first exemplary embodiment of a method for an automatic adjustment of a target position of a plug-in connecting element 32 on a patient table 17 by means of an adjusting unit 36 of an MR-PET apparatus 10. The adjusting unit 36 is herein designed in accordance with the description relating to FIG. 2.

In a first method step 100, a provision of a current position information item for the plug-in connecting element 32 takes place. The provision of the current position information item takes place by means of the position detector 40 which detects the current position of the plug-in connecting element 32. In particular, the provision of the current position information item takes place automatically and/or independently by means of the position detector 40, wherein the position detector 40 is controlled by the controller 41. The current position information item is therein provided by the position detector 40 of the controller 41.

In a second method step 101 subsequent thereto, a provision of at least one examination parameter takes place. The at least one examination parameter can therein comprise a position of the patient table 17 which it is to assume during the MR-PET examination. Alternatively, or additionally, the at least one examination parameter can also comprise a position and/or a type and/or a number of the external apparatuses 34 connected to the at least one plug-in connecting element 32, in particular, local high frequency coils. Alternatively, or additionally, the at least one examination parameter can also comprise an FOV 35 and/or a position of the beam path of the PET apparatus 12 in relation to an examination position of the patient table 17 and/or further parameters deemed useful by a person skilled in the art. The provision of the at least one examination parameter may take place automatically and/or independently by way of the controller 41 of the adjusting unit 36. Herein, the controller 41 can access examination parameters stored in the MR-PET apparatus 10.

Subsequently, in a further, third method step 102, establishing the target position of the plug-in connecting element 32 takes place on the basis of the current position information item and the at least one examination parameter. The establishment of the target position therein takes place automatically and/or independently by means of the controller 41. The target position is therein may be situated outside a beam path and/or the FOV 35 of the PET detector 12.

In a fourth method step 103 subsequent thereto, an adjustment of the target position of the plug-in connecting element 32 takes place. In particular, the adjustment of the target position of the plug-in connecting element 32 takes place automatically and/or independently by means of the motor unit 39, wherein the motor unit 39 is controlled by the controller 41.

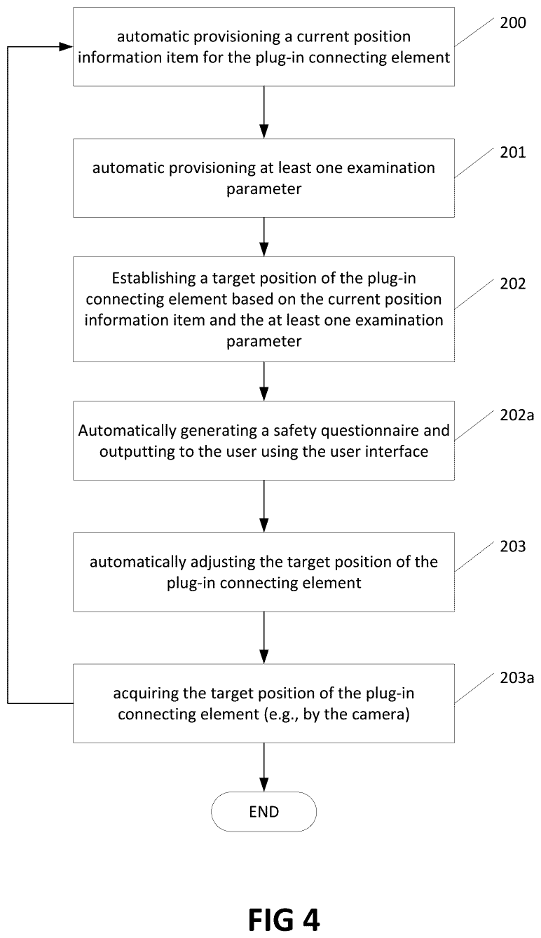

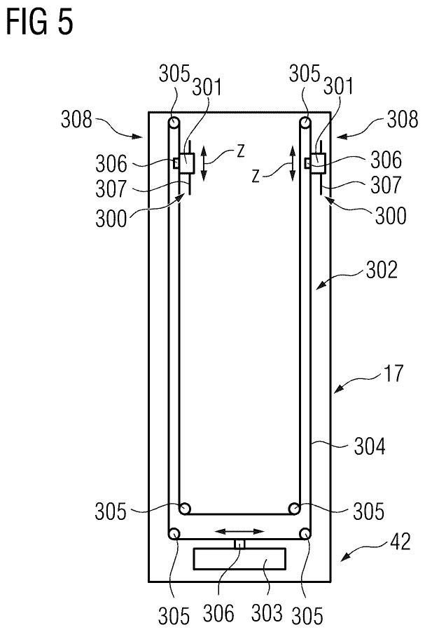

FIG. 4 shows an alternative exemplary embodiment of the method for an automatic adjustment of a target position of a plug-in connecting element 32 on a patient table 17 by means of an adjusting unit 36 of an MR-PET apparatus 10. In principle, components, features and functions that remain substantially the same are identified with the same reference signs. The following description is essentially restricted to the differences from the exemplary embodiment relating to FIG. 3, wherein with regard to components, features and functions which remain the same, reference can be made to the description of the exemplary embodiment shown in FIG. 3.

In a first method step 200, an automatic provision of a current position information item for the plug-in connecting element 32 takes place, as previously described in relation to method step 100 in FIG. 3.

In a second method step 201 subsequent thereto, an automatic provision of at least one examination parameter takes place, as previously described in relation to method step 101 in FIG. 3.

In a third method step 202 subsequent thereto, an establishment of a target position of the plug-in connecting element 32 takes place on the basis of the current position information item and the at least one examination parameter, as previously described in relation to method step 102 in FIG. 3.

In a method step 202a subsequent thereto, a safety questionnaire is automatically generated and output to the user by means of the user interface 28. The safety questionnaire is therein automatically generated by the controller 41 and is transmitted to the user interface 28, in particular the output unit 29, and is output there. The safety questionnaire can communicate to the user the established target position of the plug-in connecting element 32. In addition, the safety questionnaire comprises a user input which the user must undertake in order to start a subsequent positioning procedure. This enables the user to monitor the positioning procedure.

In a method step 203 subsequent thereto, an automatic adjustment of the target position of the plug-in connecting element 32 takes place. In particular, the adjustment of the target position of the plug-in connecting element 32 takes place automatically and/or independently by means of the motor unit 39, wherein the motor unit 39 is controlled by the controller 41.

The adjustment of the target position of the plug-in connecting element 32 by means of the motor unit 39 is therein detected (e.g., captured or otherwise determined) in a further method step 203a by a camera and is output to the user by means of the user interface 28, in particular the output unit 29. The camera can herein be included by the adjusting unit 36. In addition, for this purpose, a camera which has already been used by the MR-PET apparatus 10 for patient monitoring can also be used. The acquisition of the adjustment procedure of the target position of the plug-in connecting element 32 by the camera is herein controlled by the controller 41.

Furthermore, in this method step 203a of acquiring the target position of the plug-in connecting element 32 by means of the camera, also generated by the controller 41 is an input window, for example a pop-up window, which is displayed and/or represented for the user only during the adjustment of the target position of the plug-in connecting element 32. This pop-up window therein comprises an emergency stop switch in the form of an emergency stop button which is actuated, for example clicked, by the user in an emergency. By actuating the emergency stop button, the adjustment procedure can be ended manually by the user. This is advantageous if, for example, the user recognizes a hindrance at the target position of the plug-in connecting element 32 or an emergency situation of the patient 15 in the camera data or suchlike.

If the user does not actuate the emergency stop button in the method step 203a, following the adjustment procedure for adjusting the plug-in connecting element 32, the method for automatic adjustment of a target position of at least one plug-in connecting element 31 on a patient table 17 is ended by means of an adjusting unit 36 of an MR-PET apparatus 10.

If the user actuates the emergency stop button in the method step 203a, this leads to an immediate ending of the adjustment procedure for adjusting the plug-in connecting element 32. After a clearance of the hindrance, the method for an automatic adjustment of a target position of at least one plug-in connecting element 32 on a patient table 17 can be restarted by means of an adjusting unit 36 of an MR-PET apparatus 10.

Although the disclosure has been illustrated and described in detail by way of the exemplary embodiments, the disclosure is not restricted by the examples disclosed and other variations can be derived therefrom by a person skilled in the art without departing from the protective scope of the disclosure.

To enable those skilled in the art to better understand the solution of the present disclosure, the technical solution in the embodiments of the present disclosure is described clearly and completely below in conjunction with the drawings in the embodiments of the present disclosure. Obviously, the embodiments described are only some, not all, of the embodiments of the present disclosure. All other embodiments obtained by those skilled in the art on the basis of the embodiments in the present disclosure without any creative effort should fall within the scope of protection of the present disclosure.

It should be noted that the terms “first”, “second”, etc. in the description, claims and abovementioned drawings of the present disclosure are used to distinguish between similar objects, but not necessarily used to describe a specific order or sequence. It should be understood that data used in this way can be interchanged as appropriate so that the embodiments of the present disclosure described here can be implemented in an order other than those shown or described here. In addition, the terms “comprise” and “have” and any variants thereof are intended to cover non-exclusive inclusion. For example, a process, method, system, product or equipment comprising a series of steps or modules or units is not necessarily limited to those steps or modules or units which are clearly listed, but may comprise other steps or modules or units which are not clearly listed or are intrinsic to such processes, methods, products or equipment.

The various components described herein may be referred to as “units,” “modules,” or “devices.” Such components may be implemented via any suitable combination of hardware and/or software components as applicable and/or known to achieve their intended respective functionality. This may include mechanical and/or electrical components, processors, processing circuitry, or other suitable hardware components, in addition to or instead of those discussed herein. Such components may be configured to operate independently, or configured to execute instructions or computer programs that are stored on a suitable computer-readable medium. Regardless of the particular implementation, such units or devices, as applicable and relevant, may alternatively be referred to herein as “circuitry,” “controllers,” “processors,” or “processing circuitry,” or alternatively as noted herein.

References in the specification to “one embodiment,” “an embodiment,” “an exemplary embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

The exemplary embodiments described herein are provided for illustrative purposes, and are not limiting. Other exemplary embodiments are possible, and modifications may be made to the exemplary embodiments. Therefore, the specification is not meant to limit the disclosure. Rather, the scope of the disclosure is defined only in accordance with the following claims and their equivalents.

Embodiments may be implemented in hardware (e.g., circuits), firmware, software, or any combination thereof. Embodiments may also be implemented as instructions stored on a machine-readable medium, which may be read and executed by one or more processors. A machine-readable medium may include any mechanism for storing or transmitting information in a form readable by a machine (e.g., a computer). For example, a machine-readable medium may include read only memory (ROM); random access memory (RAM); magnetic disk storage media; optical storage media; flash memory devices; electrical, optical, acoustical or other forms of propagated signals (e.g., carrier waves, infrared signals, digital signals, etc.), and others. Further, firmware, software, routines, instructions may be described herein as performing certain actions. However, it should be appreciated that such descriptions are merely for convenience and that such actions in fact results from computing devices, processors, controllers, or other devices executing the firmware, software, routines, instructions, etc. Further, any of the implementation variations may be carried out by a general-purpose computer.

For the purposes of this discussion, the term “processing circuitry” shall be understood to be circuit(s) or processor(s), or a combination thereof. A circuit includes an analog circuit, a digital circuit, data processing circuit, other structural electronic hardware, or a combination thereof. A processor includes a microprocessor, a digital signal processor (DSP), central processor (CPU), application-specific instruction set processor (ASIP), graphics and/or image processor, multi-core processor, or other hardware processor. The processor may be “hard-coded” with instructions to perform corresponding function(s) according to aspects described herein. Alternatively, the processor may access an internal and/or external memory to retrieve instructions stored in the memory, which when executed by the processor, perform the corresponding function(s) associated with the processor, and/or one or more functions and/or operations related to the operation of a component having the processor included therein.

In one or more of the exemplary embodiments described herein, the memory is any well-known volatile and/or non-volatile memory, including, for example, read-only memory (ROM), random access memory (RAM), flash memory, a magnetic storage media, an optical disc, erasable programmable read only memory (EPROM), and programmable read only memory (PROM). The memory can be non-removable, removable, or a combination of both.

1. A magnetic resonance-positron emission tomography (MR-PET) apparatus, comprising:
a patient table; and
a plug-in connecting unit having an adjuster and at least one plug-in connector arranged on the patient table, the at least one plug-in connector being configured to enter into a releasable connection with a corresponding plug-in connector of an external apparatus,
wherein a position of the at least one plug-in connector is adjustable, the adjuster being configured to independently adjust a target position of the at least one plug-in connector on the patient table.

2. The MR-PET apparatus as claimed in claim 1, wherein the adjuster has a motor connected to the at least one plug-in connector.

3. The MR-PET apparatus as claimed in claim 2, wherein the motor is arranged at a foot end of the patient table.

4. The MR-PET apparatus as claimed in claim 2, wherein the adjuster has a guide in which the at least one plug-in connector of the plug-in connecting unit is movably mounted, the motor having a transfer element configured to displace the at least one plug-in connector within the guide.

5. The MR-PET apparatus as claimed in claim 4, wherein the transfer element is configured to displace the at least one plug-in connector by at least 1.5 mm and by not more than 40 cm within the guide.

6. The MR-PET apparatus as claimed in claim 2, wherein the adjuster has a guide in which the at least one plug-in connector of the plug-in connecting unit is movably mounted, the motor including a transfer element configured to adjust at least two different target positions of the at least one plug-in connector within the guide.

7. The MR-PET apparatus as claimed in claim 4, wherein the motor has a force sensor configured to detect a tensile force acting upon the transfer element.

8. The MR-PET apparatus as claimed in claim 1, wherein the adjuster has a position detector configured to detect a position of the at least one plug-in connector.

9. The MR-PET apparatus as claimed in claim 8, wherein the adjuster has a guide in which the at least one plug-in connector is movably mounted, the position detector being arranged on the guide.

10. The MR-PET apparatus as claimed in claim 1, wherein the adjuster has a controller configured to determine a target position for the at least one plug-in connector based on current position data relating to the at least one plug-in connector and at least one examination parameter.

11. The MR-PET apparatus as claimed in claim 10, wherein the controller is configured to control an automatic adjustment of the target position of the at least one plug-in connector.

12. A method for an automatic adjustment of a target position of at least one plug-in connector on a patient table by an adjusting unit of a magnetic resonance-positron emission tomography (MR-PET) apparatus, the method comprising:
providing current position information of the at least one plug-in connector, a position of the at least one plug-in connector being adjustable,
providing at least one examination parameter,
determining the target position of the at least one plug-in connector based on the current position information and the at least one examination parameter, and
adjusting the target position of the at least one plug-in connector based on the determined target position.

13. The method as claimed in claim 12, wherein, before the adjustment of the target position of the at least one plug-in connector, generating a safety questionnaire and outputting the generated safety questionnaire to a user by a user interface.

14. The method as claimed in claim 12, wherein the adjustment of the target position of the at least one plug-in connector is detected by a camera and is output to a user by a user interface.

15. The method as claimed in claim 12, wherein, during the adjustment of the target position of the at least one plug-in connector, the adjustment process is able to be ended manually.

16. A non-transitory computer-readable storage medium with an executable program stored thereon, that when executed, instructs a processor to perform the method of claim 12.
